(19) United States
(12) Patent Application Publication
(10) Pub. No.: US 2022/0008706 A1
(43) Pub. Date: Jan. 13, 2022

(54) MINIMALLY INVASIVE PORT IMPLANTATION

(71) Applicant: Portal Access, Inc., Wilmington, DE (US)

(72) Inventor: Michael Gabriel TAL, Tel Aviv (IL)

(21) Appl. No.: 17/146,253

(22) Filed: Jan. 11, 2021

Related U.S. Application Data
(63) Continuation-in-part of application No. PCT/US2020/041140, filed on Jul. 8, 2020.
(60) Provisional application No. 63/123,028, filed on Dec. 9, 2020.

(51) Int. Cl. A61M 39/02

(52) U.S. Cl. CPC A61M 39/0208

(57) ABSTRACT

Disclosed are a port and methods of implanting thereof minimally invasively in a body of a subject. A rear portion of the port comprises a port gripping portion configured for clamping by a clamping head of a medical clamp. The port can be pushed with the medical clamp through a surgical opening and a subcutaneous void and/or passage to a target implantation site, and, and the medical clamp can be released from the port gripping portion and removed the from the subcutaneous void and/or passage.

### RELATED APPLICATIONS

This application claims priority to PCT Application No. PCT/US20/41140, filed on Jul. 8, 2020, and to U.S. Provisional Patent Application No. 63/123,028, filed on Dec. 9, 2020; the entire contents of each of which are hereby incorporated by reference herein. The entire disclosures of all the related applications set forth in this section are hereby incorporated by reference in their entireties.

### BACKGROUND

The present disclosure relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of minimally invasive delivery and deployment thereof in a body of a subject.

Repeated needle pricking for facilitating delivery or withdrawal of fluids (e.g., medication or agents) to patient's vascular system causes harm to local tissues and decreases target blood vessel functionality and needle placement accuracy. This phenomenon is often evident in chronic diabetes, dialysis or chemotherapy patients, for example, who require continuous and repeated intravenous fluids administration for prolonged periods.

A vascular access port is a device that enables such repeated pricking and fluid administration while minimizing the accumulated harm caused by needle pricking and powered injections of fluid. The access port is subcutaneously implanted, in a surgically formed pocket in proximity to a large blood vessel, usually in the chest. It is basically formed of a port body enclosing a cavity, which is capped with a septum member configured for supporting the upper skin layers and for accepting repeated needle pricking therethrough for intravascular fluid deliveries sealed to the surrounding body tissues. The port is attached to a catheter (a thin, flexible tube) which provides fluid communication with a large blood vessel, such as the superior vena cava, in order to allow the injected fluid to dilute in the blood stream.

The implantation of a port is considered a minor procedure performed under local or general anesthesia by an interventional radiologist or a surgeon. First, the surgeon achieve access to the desired vein, a skin incision is made afterwards in the access point. Second larger incision is made above the desired location of the port, through which a pocket-like subcutaneous void is made using blunt device. The catheter is extended subcutaneously between the two incisions using a blunt tunneler. One end of the catheter is then inserted into the vein and its other end is coupled to the port. Optionally, during deployment the catheter is cut to a desired length.

Besides progress made in past years in access ports design, there is still a need to develop ports and methods of implantation and deployment thereof, which are less traumatic and invasive, and simpler to perform, potentially also by non-surgical medical personnel.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

The present disclosure relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of minimally invasive delivery and deployment thereof in a body of a subject.

In certain embodiments, there is provided a subcutaneous port. The subcutaneous port can comprise a port body enclosing a cavity, wherein the cavity comprises a first opening covered by a septum configured for repeated needle penetrations therethrough and a second opening configured for facilitating fluid communication between the cavity and a catheter. In some embodiments, the port body comprises a rigid port gripping portion configured to releasably engage with a medical clamp, and wherein the port body is configured to be pushed into a subcutaneous target implantation site using the medical clamp when the medical clamp is engaged with the port gripping portion. In some embodiments, the port gripping portion is configured to receive manual forces and/or torques from the medical clamp in at least one axis, wherein the manual forces and/or torques received at the port gripping portion are sufficient to releasably affix the medical clamp to the port gripping portion.

In some embodiments, the port body includes a rigid port body member surrounding the cavity and/or defining the first cavity opening, the rigid port body member comprising a front portion, a rear portion, and lateral portions extending from opposing sides thereof between the front portion and the rear portion, wherein the rear portion comprises the port gripping portion.

In some embodiments, the manual forces and/or torques received at the port gripping portion are sufficient to form or enlarge a subcutaneous void and/or passage in a body of a subject using the subcutaneous port and/or to maneuver the subcutaneous port along the subcutaneous void and/or passage, without slipping from, or releasing grip of, the port gripping portion.

In some embodiments, the port gripping portion comprises a wall, wherein the wall comprises opposing first and second outer wall surfaces sized to accommodate clamping surfaces of the medical clamp.

In some embodiments, the port gripping portion is configured such that the manual forces are equal to or smaller than 10 kgf and/or the manual torques are equal to or smaller than about 0.25 N*m, when the clamping surfaces of the medical clamp are oriented and spaced apart with each other to substantially match a shape and a thickness of the wall.

In some embodiments, the port gripping portion is configured such that manually operable arms of the medical clamp are allowed to interlock when the clamping surfaces of the medical clamp are oriented and spaced apart with respect to each other to match a shape and a thickness of the wall.

In some embodiments, the first and second outer surfaces are parallel.

In some embodiments, the port gripping portion comprises a clamp engagement feature.

In some embodiments, the clamp engagement feature comprises one or more of tapered surfaces, roughened surfaces, scored surfaces, teeth, recesses, and through-holes.

In some embodiments, the clamp engagement feature is formed in a wall comprising first and second outer surfaces.

In some embodiments, each of the outer wall surfaces extends vertically between lateral portions of the port body.

In some embodiments, each of the outer wall surfaces extends horizontally between a bottom portion and a top portion of the port body.

In some embodiments, an average or maximal thickness of the wall is between about 1 mm and about 4 mm, and/or an angle formed between the first and second outer wall surfaces is equal to or smaller than about 20°.

In some embodiments, the second cavity opening is in juxtaposition with, and/or located inferiorly to, the port gripping portion.

In some embodiments, the port body comprises a plurality of components coupled to each other, and wherein the port gripping portion is associated with a first one of the plurality of coupled components.

In some embodiments, the septum is associated with a second one of the plurality of coupled components different from the first one of the plurality of coupled components.

In some embodiments, the second cavity opening is associated with a third one of the plurality of coupled components different from the first and second ones of the plurality of coupled components.

In certain embodiments, there is provided a method that can comprise: forming a surgical opening across skin layers in a subject; creating a subcutaneous void and/or passage beneath the skin layers via the surgical opening; clamping a port gripping portion of a subcutaneous port with a medical clamp; pushing the subcutaneous port with the medical clamp through the surgical opening and the subcutaneous void and/or passage to a target implantation site; releasing the medical clamp from the port gripping portion; and removing the medical clamp from the subcutaneous void and/or passage.

In some embodiments, the method comprises creating or enlarging the subcutaneous void and/or passage with the medical clamp prior to the clamping.

In some embodiments, the port gripping portion comprises a wall comprising opposing first and second outer wall surfaces, wherein the clamping comprises interlocking manually operable arms of the medical clamp so as to apply continuous grip against the first and second wall surfaces of the port gripping portion.

In some embodiments, the method comprises forming the surgical opening at an axilla of the subject.

In some embodiments, the method comprises inserting a first end of a catheter to vasculature of the subject via the surgical opening and coupling a second end of the catheter to the subcutaneous port to form fluid communication between a lumen of the catheter and a cavity of the subcutaneous port.

In some embodiments, the first end of the catheter is inserted to the vasculature via axillary vein or jugular vein of the subject.

In some embodiments, any access to the vasculature and/or across the skin layers of the subject after the forming is made directly through the surgical opening.

In some embodiments, the method further comprising at least one of: accessing into a vein of the subject with an access needle, inserting a wire into the vein through the access needle, removing the access needle from the vein, inserting a peel apart sheath and/or a dilator into the vein over the wire, removing the wire and/or the dilator from the vein, inserting a first end of a catheter into the vein through the peel apart sheath, and removing the peel apart sheath from the vein.

In some embodiments, the vein is an axillary vein or a jugular vein.

In some embodiments, the medical clamp is configured as medical forceps and/or selected from Kelly forceps, a surgical needle holder, and locking forceps.

In certain embodiments, there is provided a kit for creating repeatable therapeutic access to a subject. The kit can comprise: a subcutaneous port, the subcutaneous port comprising a port body enclosing a cavity, wherein the cavity comprises a first opening covered by a septum configured for repeated needle penetrations therethrough and a second opening configured for facilitating fluid communication between the cavity and a catheter; and a medical clamp. In some embodiments, the port body comprises a port gripping portion configured to releasably engage with the medical clamp, and wherein the port body is configured to be pushed into a subcutaneous target implantation site using the medical clamp when the medical clamp is engaged with the port gripping portion.

In some embodiments, the medical clamp comprises opposing pivotally connected clamping arms.

In some embodiments, the port gripping portion comprises a wall, wherein the wall comprises first and second outer surfaces sized to accommodate distal portions of the opposing pivotally connected clamping arms.

All technical or/and scientific words, terms, or/and phrases, used herein have the same or similar meaning as commonly understood by one of ordinary skill in the art to which the invention pertains, unless otherwise specifically defined or stated herein. Illustrative embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIGS. 1A-1C schematically illustrate respectively a side cross-sectional view and a top cross-sectional view of an exemplary deployed vascular access port, in accordance with some embodiments;

FIGS. 2A-2C schematically illustrate different exemplary variations of a port gripping portions of the exemplary subcutaneous port shown in FIG. 1A, according to some embodiments;

FIGS. 3A-3H schematically illustrate exemplary scenarios representing steps in an exemplary procedure for implanting the exemplary subcutaneous port shown in FIG. 1A, according to some embodiments;

FIGS. 4A-4J schematically illustrate exemplary scenarios representing steps in an exemplary procedure for implanting the exemplary subcutaneous port shown in FIG. 1A via a single opening formed at subject's axilla, according to some embodiments;

FIGS. 5A-5B respectively illustrate an exemplary subcutaneous port in an assembled isometric view and in an exploded isometric view, according to some embodiments;

FIGS. 6A-6B respectively illustrate the exemplary subcutaneous port shown in FIG. 5A in a side cross-sectional view and in a frontal cross-sectional view, according to some embodiments;

FIG. 7 illustrates the exemplary subcutaneous port shown in FIG. 5A grasped with an exemplary surgical needle holder, according to some embodiments;

FIGS. 8A-8B illustrate axonometric views of another exemplary subcutaneous port comprising a port gripping portion, in accordance with some embodiments;

FIGS. 9A-9B illustrate respectively a top view and an axonometric view of the subcutaneous port of FIG. 8A clamped with medical forceps, in accordance with some embodiments; and

FIG. 10 illustrates an axonometric view of an exemplary subcutaneous port comprising another exemplary configuration of a port gripping portion, in accordance with some embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

The present disclosure, in some embodiments thereof, relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject. In some embodiments, vascular access ports of the present disclosure can improve safety and/or efficacy of the surgical implantation procedure of access port and catheter by reducing size or number of surgical processes (like cuts, incisions, and tunneling), their duration and/or complexity, thereby also provide a less traumatic experience and easier recovery for the patient.

As used herein, the term “vascular access port” refer to an implant intended for repeated transfer of fluids administered to and/or withdrawn from a subject. “Repeated” in this context may refer to more than 10 consecutive needle punctures, optionally more than 100 consecutive needle punctures, optionally more than 1,000 consecutive needle punctures, optionally more than 10,000 consecutive needle punctures, or higher or lower. “Needle” in this context may refer to needles approved for fluid deliveries through vascular access ports, such as for intravenous administration.

The disclosures described herein are advantageous also when used in conjunction with vascular access ports that have a septum member configured for repeated puncturing by a needle, but this particular feature is not a requirement and other forms of needle access openings or platforms may apply. Some vascular access ports described herein include one or more components configured, collectively, when properly assembled and deployed, for prolonged implantation in a live (e.g., human) subject and for repeated fluid transfer access, such as through a septum member. The vascular access port includes at least a structural object referred to herein as a “port body” which serves as a facilitating structure for fluid transfer access and/or as a support structure configured for holding components (e.g., a septum) applicable for fluid transfer access.

The port body may be structurally and/or functionally configured for facilitating at least the basic function of the vascular access port of repeated accumulating and delivering and/or withdrawing fluid to or from subject's vasculature. In some embodiments, the port body may optionally lack or be initially configured without one or more other features, optional or vital ones, for facilitating additional functions associated with delivery, deployment and/or prolonged use of a vascular access port. The port body may be connected to at least one other component for providing the vascular access port additional features or capabilities, for example improved or easier deliverability, selective fixation to body tissues surrounding the port body and/or increased stability in a chosen implantation site such as a preformed subcutaneous void.

In some embodiments, the port body forms a cavity beneath (e.g., inferiorly to) the needle access opening and/or septum, which is sized, shaped and configured for repeatedly receiving a needle tip, for accumulating a chosen or predetermined volume of fluid (e.g., a liquid such as a solution, a suspension or a colloid), and/or for fluid administration to, and/or withdrawal from, a vasculature of the live subject. In some embodiments, a vascular access port may include a single cavity or several distinct cavities, optionally covered with one or several distinct septum members, provided as a single element or as several interconnectable members, some or all can be provided in the port body or in several portions or members of the vascular access port configured each as a separate port body.

Before or after implantation, a catheter may be attached to the vascular access port with a distal end that physically enters the vasculature of the patient. Once connected, a lumen of the catheter is provided in direct fluid communication with the port body cavity. A vascular access port as described herein, or a kit comprising it, may include or not include such a catheter and may include or not include a fitting for connecting to such a catheter. A vascular access port may have additional components and functionality not associated with fluid delivery or withdrawal. A vascular access port may be referred to herein as simply a “port” or an “implant”. A “subcutaneous port” refers to a vascular access port and optionally, more generally, to any other medical implantable port, configured particularly for implantation beneath skin tissues and is accessible by way of needle puncture or penetration thereinside, percutaneously, through skin tissues covering it.

The vascular access port optionally includes a port gripping portion configured for facilitating effective continuous, optionally locked, clamping or grasping of the port using a medical clamp. The medical clamp includes a distal clamping head that is selectively operable using elongated arms extending distally therefrom, and the clamping head is selectively changeable between an open (non-clamping) configuration and a closed (clamping) configuration. When in the closed configuration, two clamping surfaces of two opposing pivotally-connected clamping head members forming the clamping head, are pressed against each other from both sides of the port gripping portion. The medical clamp may be provided to the user together with the port, optionally as a kit, or it can be a generic clamping or grasping device commonly used by medical practitioners performing subcutaneous port implantations, optionally configured as medical forceps, such as Kelly forceps, surgical needle holder, surgical graspers, locking forceps, hemostat, or another.

Deploying the vascular access port includes at least inserting the port body into a target implantation site in the subject body, such that a superior portion of the port body is accessible to repeated fluid transfer access. Insertion of the port (or port body) can be performed using the medical clamp by first applying it to continuously clamp (and optionally lock it in this clamping position) the port gripping portion, and then manually push and/or maneuver the port using the medical clamp arms.

Vascular access port deployment may include compacting of tissue mass surrounding periphery of the port body thereby increasing a volume of a void formed in the target implantation site between the periphery of the port body and the compacted tissue mass. In some embodiments, the void and/or a surgical passage thereto from an incision on subject's skin, can be performed using the same medical clamp (e.g., Kelly forceps or needle holder) before it is clamped to the port. The void can be a subcutaneous void located between or beneath skin tissue layers at the target implantation site. Concurrently with increasing the void volume, or immediately afterwards, the increased void volume is occupied with the vascular access port such as by increasing the volume of the port body or by connecting one or more solid shaped components (e.g., a port body extension) thereto. This also includes the situation that the tissue mass compaction may be a direct result of such increase in port body volume. The compacted tissue mass normally affects a continuous pressure on the deployed vascular access port and thereby increase its fixation and/or stability in the subcutaneous void. The port body may include an inferior portion which defines a cavity and a superior portion coupled with a septum member covering the cavity, and the vascular access port may be deployed such that the compacted tissue mass surrounds only an inferior portion and not the superior portion of the port body.

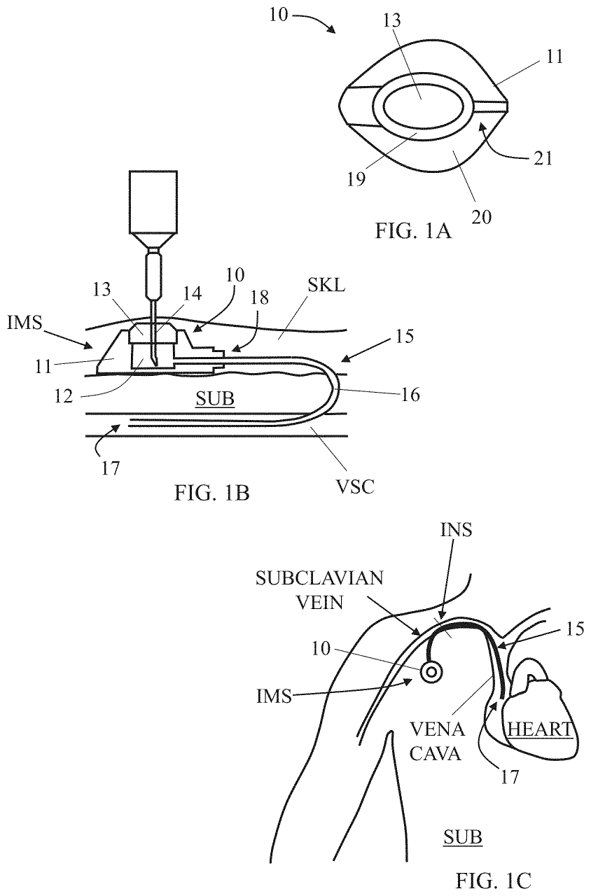

FIGS. 1A-1C schematically illustrate an exemplary vascular access port 10, configured as a subcutaneous port, before and after implantation in a subject SUB (e.g., a live human patient). Vascular access port 10, as shown in top view in FIGS. 1A and 1n side-cut view in FIG. 1B, includes a port body 11 defining a cavity 12 and coupled with a septum member 13 that covers and seals cavity 12 from surroundings. Septum member 13 is configured for repeated puncturing of needles, like needle 14 shown in FIG. 1B, without compromising sealing of cavity 12 during needle placement therethrough and after the needle is withdrawn. Cavity 12 is optionally opened to a first cavity opening that is enclosed with septum member 13 and to a second cavity opening configured for facilitating fluid communication between the cavity and a lumen of a catheter when connected thereto. In some embodiments, the second cavity opening is located at a rear portion of port 10, in juxtaposition with, and/or located inferiorly to, a port gripping portion 21 configured for gasping or clamping by a medical clamp.

Port 10 is implantable in a target implantation site IMS subcutaneously beneath skin layers SKL (including optionally within or beneath fat tissue) via a single opening or incision INS in a subject SUB. When fully deployed, vascular access port 10 has cavity 12 in fluid communication with vasculature VSC of subject SUB, normally a large blood vessel such as the Subclavian vein or one of the Vena Cavae, so that fluid administrated into cavity 12 via needle 14 will flow directly to the subject's vascular system. A catheter 15 with catheter lumen 16 has a first catheter end 17 thereof positioned in and opened to vasculature VSC, and a second catheter end 18 thereof is connected to port body 11 and opened to cavity 12; catheter ends, 17 and 18, are opened to catheter lumen 16 and facilitate fluid communication between cavity 12 and vasculature VSC. In some embodiments, both port 10 and catheter 15 are introduced and implanted in subject SUB via the single opening or incision INS. FIG. 1B shows an optional deployment scheme where port 10 is positioned on an upper part the subject's chest in proximity to access opening made to Jugular vein, with first catheter end 17 positioned in the superior vena cava in proximity to subject's right atrium. Vascular access port 10 may be provided separately to catheter 15 with a connector configured for selective connection therebetween, optionally within the body, or alternatively vascular access port 10 and catheter 15 are provided together as an assembly kit or as a unified device.

In some embodiments, port 10 may be substantially rigid such that most or all portions thereof surrounding cavity 12 are not deformable or shapeable under normal stresses originating during or after implantation subcutaneously in the subject's body, and in some other embodiments, at least one portion there if designed and configured for flexing or moving relative to other portions of port 10 (e.g., relative to port body 11 or rigid portions thereof) before, during or after implantation. In some embodiments, port 10 is configured as a squeezable subcutaneous port capable of penetrating through a small opening, such as one formed by puncture or incision made to patient's skin, incapable of accommodating passage therethrough of port 10 in its maximal cross-sectional circumference when in an elastically relaxed state. Penetration through such an opening can be accomplished by forcing one or more portions of port 10 to elastically compress locally by the opening neck portion, when it is pushed distally through the opening.

In some such embodiments, port 10, and particularly port body 11, includes a rigid inner member 19, which forms cavity 12, and a flexible outer member 20 connected to inner member 19 along at least one lateral periphery portion of the inner member, thereby forming a chosen predetermined spatial shape for port 10 (as shown in FIG. 1A, for example) when in an elastically relaxed state. Outer member 20 may be configured with elastic resistance to compression sufficient to maintain the predetermined spatial shape within a surgically formable subcutaneous void when under naturally occurring subcutaneous stresses. Furthermore, outer member 20 is locally compressible against inner member 19, and configured to substantially maintain an overall volume by enlarging remotely to a compressed region thereof, thereby facilitating squeezing of port 10 into the subcutaneous void when pushed through a skin opening greater than a maximal cross sectional circumference of the inner member and smaller than a maximal cross sectional circumference of the predetermined spatial shape.

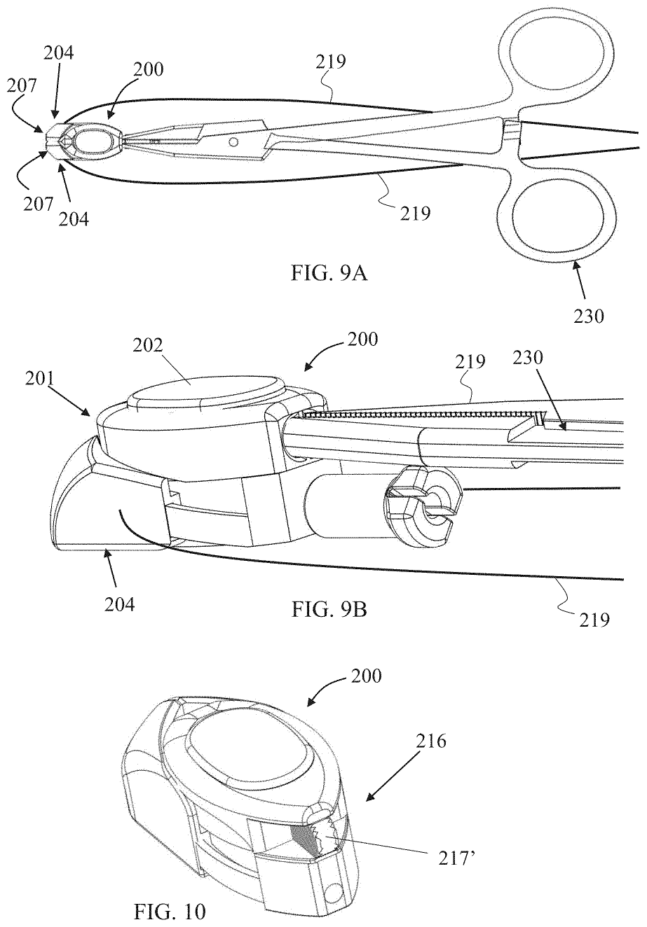

Port 10 includes a port gripping portion 21 having a relatively thin wall bordered with two opposing surfaces configured for effective continuous clamping or grasping by a medical clamp (such as surgical needle holder 119 shown in FIG. 7 and/or Kelly forceps 230 shown in FIG. 9A, for example). Port gripping portion 21 is located at or extended from rear portion of body of port 10, or a rigid structural member thereof, and optionally includes a thin wall bounded by two opposing surfaces sized and shaped for effective clamping by the medical clamp. In some embodiments, an average or maximal thickness of the thin wall is between about 1 mm and about 4 mm, or between about 1.5 mm and about 3 mm. In some embodiments, an angle formed between the opposing surfaces of the thin wall is equal to or smaller than about 20°, optionally equal to or smaller than about 15°, or optionally equal to or smaller than about 10°.

Port body 11 may include a superior portion or member which encloses at least superior portion of cavity 12 and/or contacts septum member 13, and this superior portion or member may narrow at rear portion thereof to form port gripping portion 21. Port gripping portion 21 is configured with sufficient rigidity and/or strength for preventing mechanical failure, when clamped and manually maneuvered beneath skin layers of subject SUB, and is optionally made of metal alloy such as stainless steel or titanium alloy or hard polymer such as polyether ether ketone (PEEK). In some embodiments, port gripping portion 21 is shaped with opposing surfaces thereof so as to accommodate a desired clamping orientation of medical clamp thereto.

Port gripping portion 21 is configured for transferring manual forces and/or torques through the medical clamp to port 10 in at least one axis, for example a first axis of grasping normal to the thin wall surfaces and optionally other axes in other directions. The manual forces and/or torques are optionally sufficient to lock the medical clamp to port gripping portion 21. In some embodiments, the manual forces and/or torques are also sufficient to form or enlarge a subcutaneous void and/or passage in the subject's body using clamped port 10 and/or to maneuver port 10 along the subcutaneous void and/or passage, without slipping from, or releasing grip of, the port gripping portion 21. Port gripping portion 21 is optionally configured such that the manual forces are equal to or smaller than about 20 kgf, optionally equal to or smaller than about 10 kgf, equal to or smaller than about 5 kgf, when the distal portions of the clamping head members forming medical clamp are oriented and spaced apart with each other to match shape and thickness of the thin wall. Port gripping portion 21 is optionally configured such that the manual torques are equal to or smaller than about 0.25 N*m, equal to or smaller than about 0.20 N*m, or equal to or smaller than about 0.15 N*m, when the distal portions of the clamping head members forming medical clamp are oriented and spaced apart with each other to match shape and thickness of the thin wall.

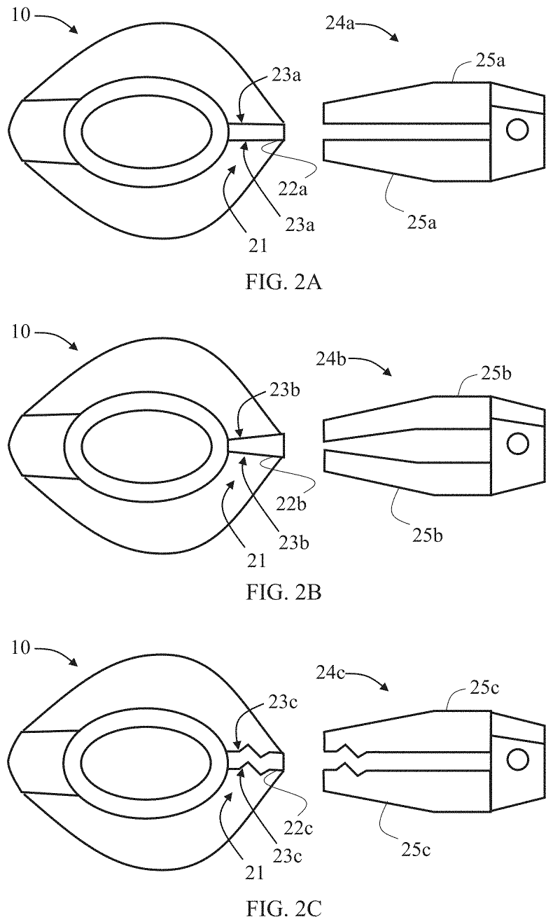

FIGS. 2A-2C schematically illustrate different exemplary variations of port gripping portion 21 of port 10. FIG. 2A shows a first exemplary variation for port gripping portion 21 having a thin wall 22a with two opposing surfaces 23a being substantially flat and parallel with each other so as to effectively accommodate a first exemplary medical clamp head 24a configured for applying sufficient clamping force when clamping head members 25a thereof are oriented substantially parallel with each other, at least with distal portions thereof, when clamping head members 25a are spaced apart at a distance equal to or slightly smaller than thickness of thin wall 22a. FIG. 2B shows a second exemplary variation for port gripping portion 21 having a thin wall 22b with two opposing surfaces 23b being substantially flat and tapered with each other so as to effectively accommodate a second exemplary medical clamp head 24b configured for applying sufficient clamping force when clamping head members 25b thereof are oriented substantially tapered with each other, at least with distal portions thereof, when clamping head members 25b are spaced apart at a distance equal to or slightly smaller than thickness of thin wall 22b. FIG. 2C shows a third exemplary variation for port gripping portion 21 having a thin wall 22c with two opposing surfaces 23c having each a non-flat (e.g., toothed) pattern. As such, it can effectively accommodate a third exemplary medical clamp head 24c configured for locking thereto in at least one axis when clamping head members 25c thereof engage surfaces 23c with mating non-flat patterns thereof when they are spaced apart at a distance equal to or slightly smaller than thickness of thin wall 22c.

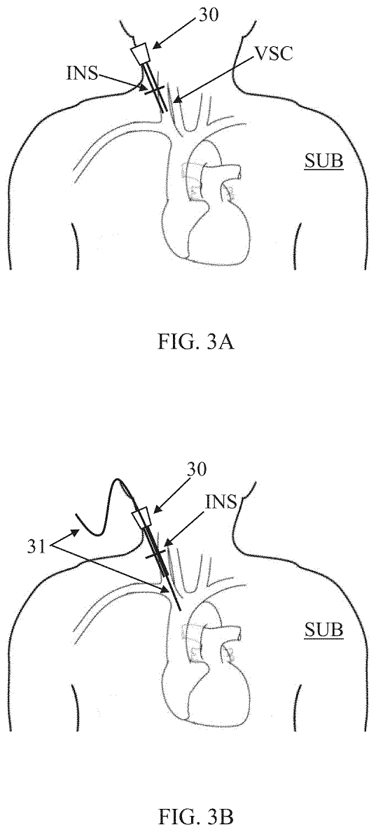

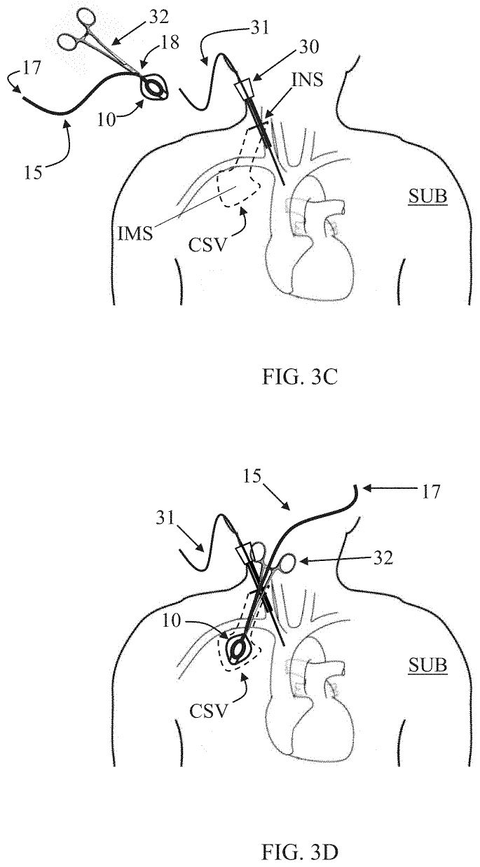

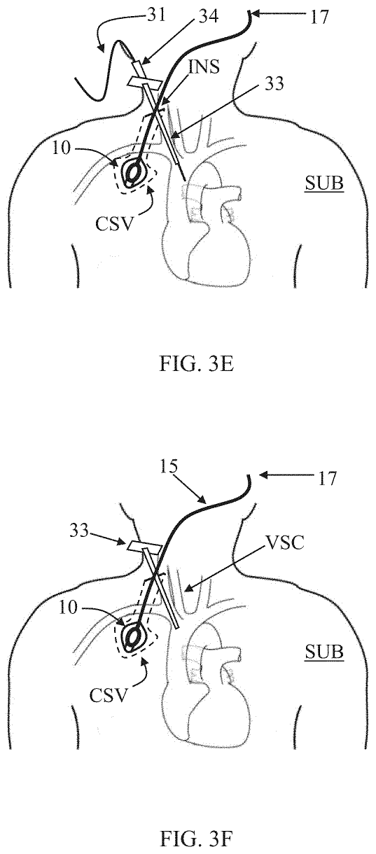

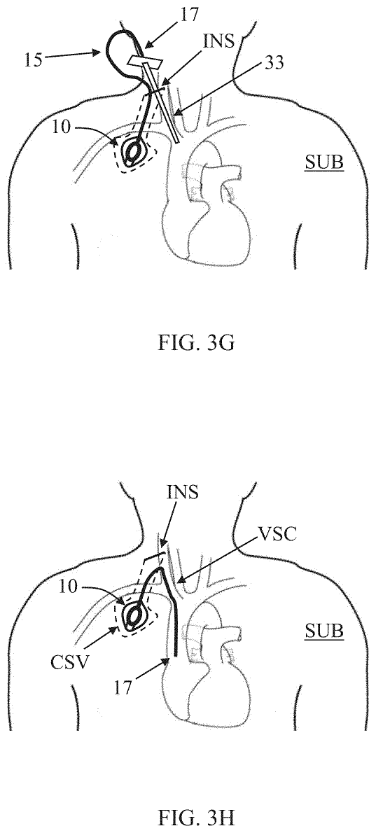

FIGS. 3A-3H schematically illustrate exemplary scenarios representing possible steps in an exemplary procedure for implanting vascular access port 10 with catheter 15 in subject SUB, via a single surgically made opening across patient's skin. The exemplary order of implantation first shows steps related to forming access into vasculature VSC followed by steps related to implantation of port 10 in a subcutaneous void in the body of subject SUB, and then other steps related to catheterization of catheter 15 by positioning first end thereof in the vasculature, however the procedure according to some embodiments can be performed in any different order such as by first implanting port 10 and then forming access and implanting catheter first end 17, implanting port 10 after catheterization, or implanting them both in parallel at least in part. As shown in FIG. 3A, a surgical opening (optionally by way of incision INS) is formed across skin layers of subject SUB, optionally at the neck area or proximately superiorly (above) to the clavicle, and an access needle 30 is introduced therethrough into nearby vasculature VSC (e.g., the jugular vein, as illustrated, or the subclavian vein). In some embodiments, access needle 30 is applied with tip thereof to puncture and/or penetrate through the skin, and thereby optionally forming the surgical opening or part thereof, optionally before an incision is made with a scalpel thereacross or adjacent thereto. As shown in FIG. 3B, a guidewire 31 can then be inserted through the lumen of access needle 30 into vasculature VSC.

As shown in FIG. 3C, port 10 can be clamped with a medical clamp 32 for assisting in its implantation. In the event incision INS is not already formed, the medical practitioner can make the incision immediately before port implantation, or increase size of incision INS with a scalpel or with port 10 itself, for example. Before clamping on to port 10, a subcutaneous void and/or passage SCV can be formed via incision INS using medical clamp 32 or with another instrumentation. The subcutaneous void and/or passage SCV extends from incision INS to target implantation site IMS which is located optionally at the upper chest area and/or inferiorly (below) to the clavicle.

Port 10 can then be pushed into subcutaneous void and/or passage SCV via incision INS using medical clamp 32 clamped thereto (FIG. 3D). In some embodiments, the presented order is reversed and port 10 is implanted at implantation site IMS before access needle 30 and/or guidewire 31 is introduced into body of subject SUB. In some embodiments, second end 18 of catheter 15 is already (fixedly or releasably) connected to port 10, as shown, however it may be provided disconnected and can be connected before or during delivery through incision INS, before or after clamping port 10 with medical clamp 32. Following implantation of port 10 at the target implantation site IMS, first end 17 of catheter 15 is optionally located outside body of subject SUB, however in some other embodiments first end 17 of catheter 15 can be put inside vasculature VSC before port 10 is implanted. Once port 10 proper position is verified, medical clamp 32 can be release (unclamped) from port 10 and withdrawn from subcutaneous void and/or passage SCV.

Before or after implantation of port 10, access needle 30 can be removed from body of subject SUB while leaving guidewire 31 in the chosen path within vasculature VSC (FIG. 3E). Following that, a peel-apart sheath 33 (e.g., that can be torn along premade weakening lines) can be inserted into vasculature VSC over guidewire 31. Guidewire 31 may then be removed from vasculature VSC leaving peel-apart sheath 33 in-place. In case peel-apart sheath 33 was introduced with a dilator 34 extending along its lumen, dilator 34 can also be withdrawn from within peel apart sheath 33, as shown in FIG. 3F. Once lumen of peel-apart sheath 33 is patent, first end 17 of catheter 15 is introduced therethrough into vasculature VSC and optionally positioned in the superior vena cava or into the right atrium, as shown sequentially in FIGS. 3G and 3H. After verifying port 10 and/or catheter 15 are in proper position and function, optionally under imaging, peel-apart sheath 33 is broken apart and removed from body of subject SUB and incision INS is closed (e.g., by way of suturing).

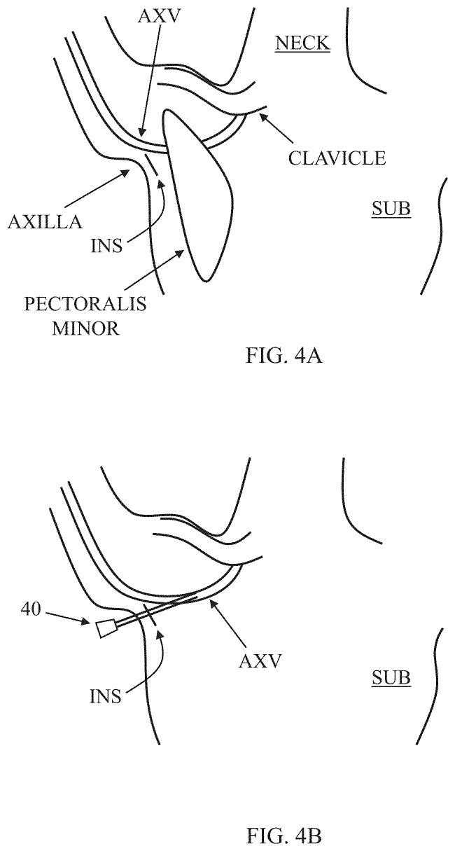

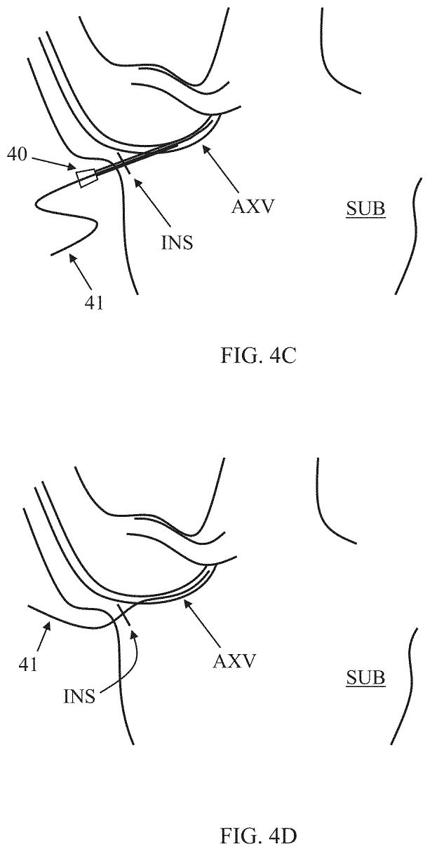

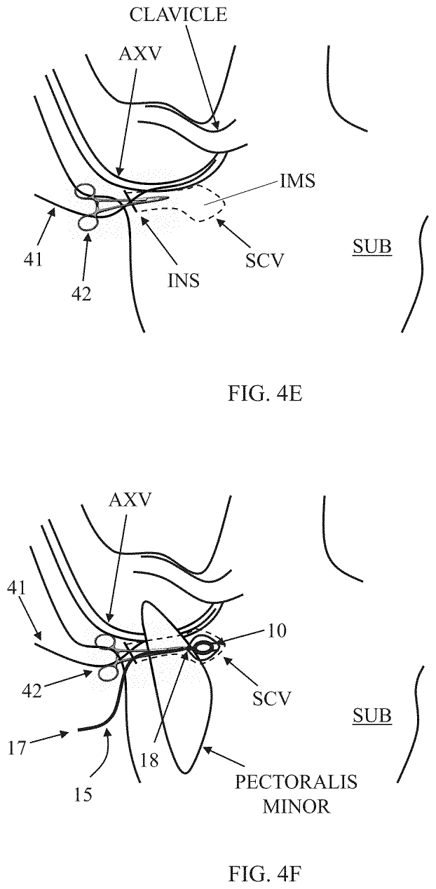

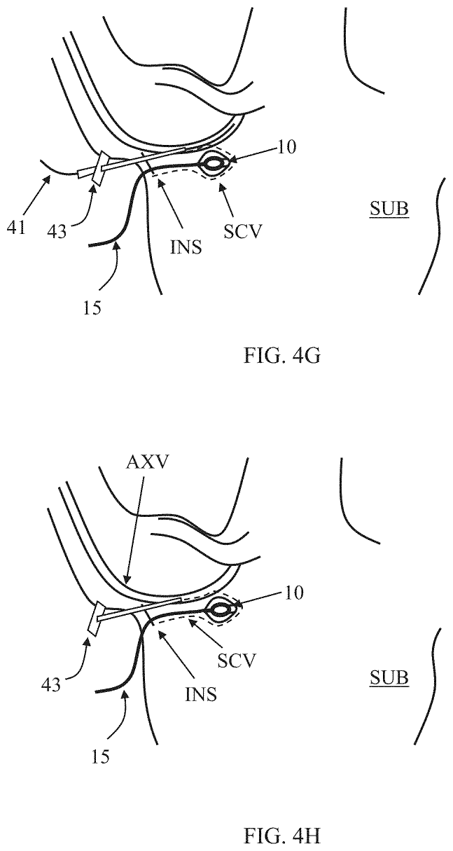

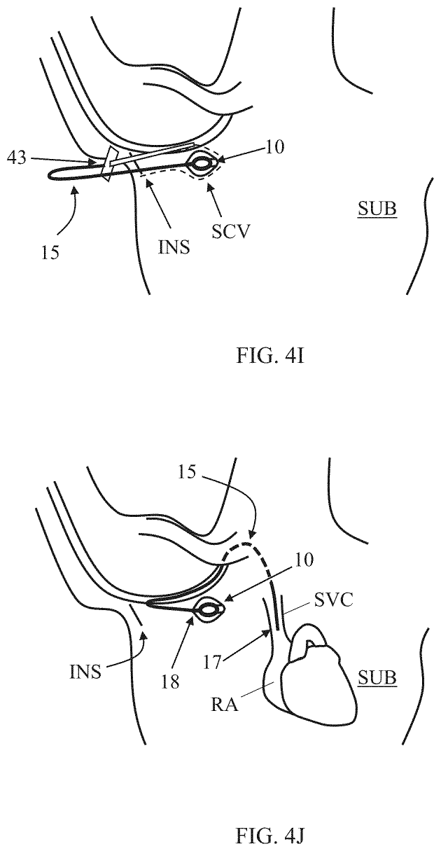

FIGS. 4A-4J schematically illustrate exemplary scenarios representing steps in an exemplary procedure for implanting vascular access port 10 with catheter 15 in subject SUB, via a single surgically made opening across patient's skin at axilla (armpit) of subject SUB. The exemplary order of implantation first shows steps related to forming access into subject's vasculature followed by steps related to implantation of port 10 in a subcutaneous void in the body of subject SUB, and then other steps related to catheterization of catheter 15 by positioning first end thereof in the vasculature, however the procedure according to some embodiments can be performed in any different order such as by first implanting port 10 and then forming access and implanting catheter first end 17, implanting port 10 after catheterization, or implanting them both in parallel at least in part.

FIG. 4A illustrates a partial in-body view of upper torso of subject SUB. A surgical opening (optionally by way of incision INS) is formed across skin layers of subject SUB at or in proximity to the axilla area in joining of the right or the left hand to the respective shoulder of subject SUB. Incision INS is formed optionally adjacent to an inferior portion of the axillary vein AXV and/or inferiorly (below) the pectoralis minor muscle. As shown in FIG. 4B, an access needle 40 can then be introduced through incision INS into axillary vein AXV, optionally in inferior portion thereof as shown. In some embodiments, access needle 40 is first applied with a tip thereof to puncture and/or penetrate through the skin, and thereby optionally forming the surgical opening or part thereof, optionally before incision INS is made (e.g., using a scalpel) thereacross or adjacent thereto. A guidewire 41 can then be inserted through lumen of access needle 40 into subject's vasculature through axillary vein AXV (FIG. 4C) and afterwards access needle 40 can be removed (FIG. 4D) leaving guidewire 41 extending in axillary vein AXV.

As shown in FIG. 4E, a subcutaneous void and/or passage SCV can be formed via incision INS using a sharp surgical instrument, optionally with a medical clamp 42 (which may be similar or identical to medical clamp 32, for example). The subcutaneous void and/or passage SCV extends from incision INS to target implantation site IMS which is located optionally at the upper chest area and/or inferiorly (below) to the clavicle, optionally beyond the pectoralis minor (as shown in FIG. 4G) and/or optionally reaching between the third rib and the clavicle, optionally adjacent to the top portion of the second rib. Port 10 can then be clamped with medical clamp 32 for assisting in its implantation. In the event incision INS is not already formed, the medical practitioner can make the incision immediately before port implantation or increase size of incision INS with a scalpel or with port 10 itself, for example.

As shown in FIG. 4F, port 10 can be pushed into subcutaneous void and/or passage SCV via incision INS, using medical clamp 32 clamped thereto, and implanted in target implantation site IMS. In some embodiments, the presented order can be reversed and port 10 is implanted at implantation site IMS before access needle 40 and/or guidewire 41 is introduced into body of subject SUB. In some embodiments, second end 18 of catheter 15 is already (fixedly or releasably) connected to port 10, as shown, however it may be provided disconnected and can be connected before or during delivery through incision INS, before or after clamping port 10 with medical clamp 42. Following implantation of port 10 at the target implantation site IMS, first end 17 of catheter 15 is optionally located outside body of subject SUB, however in some other embodiments first end 17 of catheter 15 can be put inside axillary vein AXV before port 10 is implanted.

Once port 10 proper position is verified, medical clamp 42 can be release (unclamped) from port 10 and withdrawn from subcutaneous void and/or passage SCV, as shown in FIG. 4G. Before or after implantation of port 10, a peel-apart sheath 43 can be inserted into axillary vein AXV over guidewire 41. Guidewire 41 may then be removed from subject's vasculature leaving peel-apart sheath 43 in-place (FIG. 4H). In case peel-apart sheath 43 was introduced with a dilator extending along its lumen, the dilator can also be withdrawn from within peel apart sheath 43. Once lumen of peel-apart sheath 43 is patent, first end 17 of catheter 15 is introduced therethrough into axillary vein AXV and optionally positioned in the superior vena cava SVC or into the right atrium RA, as shown sequentially in FIGS. 4I and 4J. Before insertion, catheter 15 can be cut to a chosen length optionally based on measurements of the path length from implantation site IMS to the chosen positioning of first catheter end 17 in patient's vasculature. Final positioning of catheter first tip 17 and/or port 10 can be applied by pushing or pulling port in subcutaneous void and/or passage SCV. After verifying port 10 and/or catheter 15 are in proper position and function, optionally under imaging, peel-apart sheath 43 is broken apart and removed from body of subject SUB and incision INS is closed (e.g., by way of suturing or bonding).

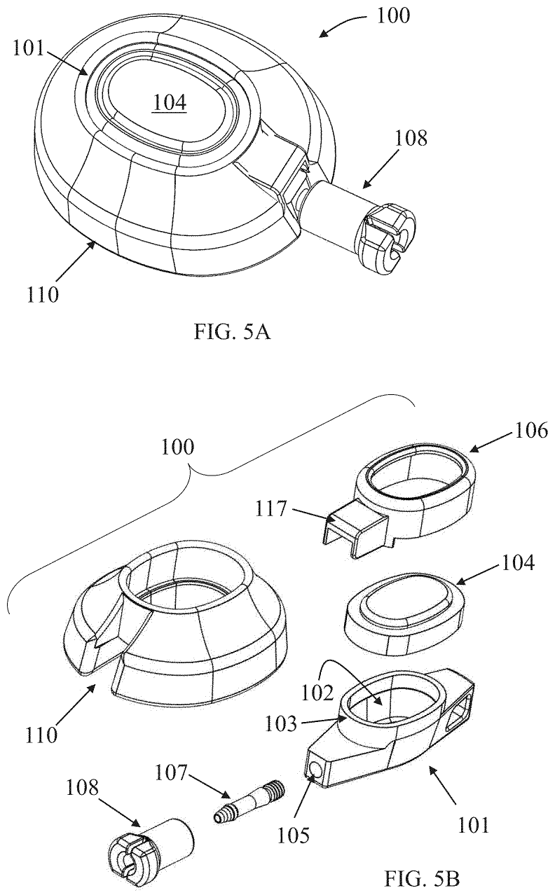

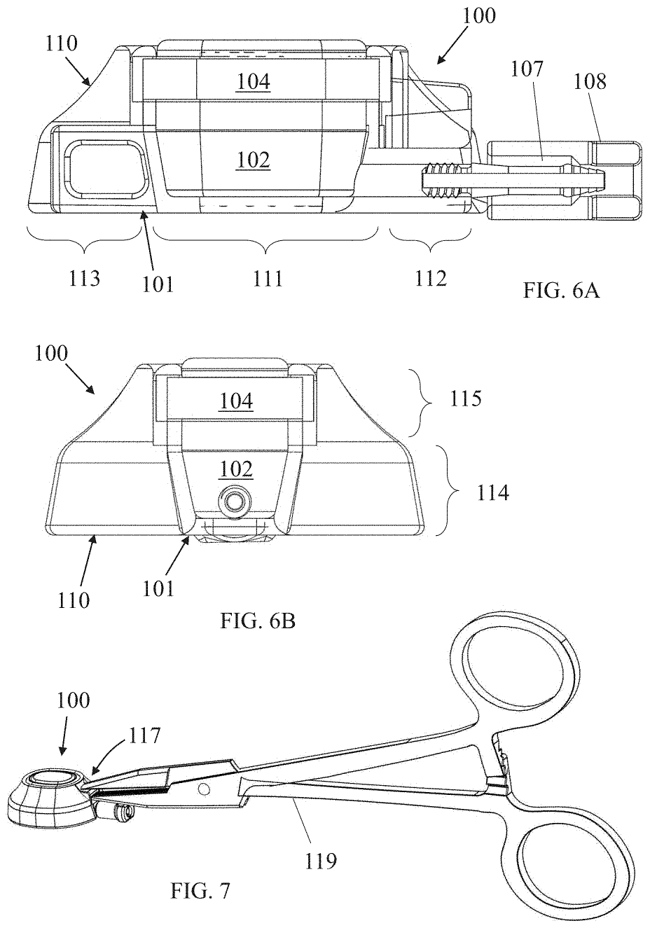

FIGS. 5A-5B respectively illustrate an exemplary squeezable subcutaneous port 100 in an assembled isometric view and in an exploded isometric view. FIGS. 6A-6B respectively illustrate port 100 in a side cross-sectional view and in a frontal cross-sectional view. Port 100 is optionally an exemplary embodiment, representation, or variation of port 10, and may include some or all structural and/or functional features described with respect to port 10. Port 100 in an elastically relaxed state may have a maximal width of 50 mm or less, optionally 25 mm or less; a maximal height of 30 mm or less, optionally 15 mm or less; and a maximal length (with or without catheter connecting means) of 50 mm or less, optionally 30 mm or less. In some embodiments, port 100 is configured for squeezing through surgical openings (without further widening or tearing when passing therethrough) having a maximal opening circumference of about 80 mm or less, optionally of about 60 mm or less, optionally of about 40 mm or less, and/or formed by a surgical incision of about 20 mm or less in length, optionally about 15 mm or less in length, or optionally about 10 mm or less in length.

Port 100 includes a rigid inner member 101 comprising a cavity 102 opened to a first cavity opening 103 and to a second cavity opening 105. First cavity opening 103 is enclosed with a septum member 104 and configured for repeated needle penetrations therethrough into cavity 102. Second cavity opening 105 is configured for facilitating fluid communication between cavity 102 and a lumen of a catheter. Inner member 101 is configured with sufficient rigidity to accommodate (safely and efficiently) a chosen length of a needle and to prevent the needle's tip from penetrating therethrough. Septum member 104 is optionally oval, as shown, although it may have any other shape.

A cap member 106 is coupled over septum member 104 and over the superior portion of inner member 101 to form a unitary rigid encapsulated core body of port 100. Septum member 104 is restrained in-position and optionally compressed, at least partly, by and in-between cap member 106 and inner member 101. Inner member 101 and/or cap member 106 are optionally formed of hard plastic such as PEEK, or from metal such as titanium or stainless-steel alloys. Cap member 106 is optionally fixedly connected to inner member 101, such as by way of adhesives, compressing fitting and/or welding (e.g., ultrasonic welding if the parts are made of plastic, or laser welding if the parts are made of metal). The encapsulated core body, once fully assembled, has sufficient rigidity and yield strength, and is configured to maintain internal pressures that are common during injections into cavity 102 (of optionally about 5 ml/sec injections at 300 psi, or higher or lower). A lumen extension 107 is coupled to inner member 101 with distal portion thereof extending towards cavity 102 through second cavity opening 105 and configured to provide a fluid-tight passage via proximal portion thereof to a catheter lumen. A connector member 108 is coupled over lumen extension 107 and is configured to facilitate selective connection of a catheter distal end with port 100, such as with a luer-fitting based connection mechanism.

Port 100 includes a port gripping portion 117 (optionally similar or identical in structure, function and/or dimensions to port gripping portion 21) provided at proximal end thereof and is configured for facilitating efficient and safe grasping of port 100 with grasping means, such as Kelly forceps or surgical needle holder. Port gripping portion 117 may be provided as a proximal extension of cap member 106, as shown, and located above (superiorly to) lumen extension 107 and connector member 108. FIG. 7 illustrates port 100 grasped at port gripping portion 117 with an exemplary surgical needle holder 119. Port gripping portion 117 is shown with its flat surface extending horizontally so that needle holder 119 can be held by the medical practitioner having its arms arranged vertically (one over the other). Alternatively, port gripping portion 117 can be arranged with its flat surface in any other direction, including optionally vertically. Port gripping portion 117 is optionally configured in size, surface area of its flat surface, thickness and/or durability and/or strength to facilitate firm grasping by needle holder 119 sufficiently to push, squeeze-in by elastic compression, and maneuver port 100 through a surgical opening smaller than its maximal relaxed dimensions, without releasing grip or mechanical failure. Needle holder 119 can be used to form or increase size of a subcutaneous void before grasping on to port 100 and delivering it into the subcutaneous void.

In some embodiments, inner member 101 can be functionally configured or applicable to serve as a vascular access port although it may be incapable, insufficient, or less compatible of providing one or more, optionally essential, features for improving, facilitating or easing implantation and/or long-term use of port 100. Port 100 includes a flexible outer member 110 which provides, at least when it is in an elastically relaxed state, a final spatial shape and size, for providing one or more additional features, including but not limited to: stability and/or fixation in implantation site, transdermal accessibility, identification and/or locating of septum member 104 for repeated percutaneous fluid administration, protection to port body and/or overlaying skin layers, or others.

Outer member 110 is connected to inner member 101 along at least one lateral periphery portion thereof, thereby forming a chosen predetermined spatial shape of the subcutaneous port when in an elastically relaxed state. Optionally, outer member is configured as a skirt or ring-like element encompassing most or all periphery of inner member 101, and optionally also periphery of cap member 106, in at least a circumferential segment thereof. In order to maintain sufficient rigid pushability of port 100 for its insertion and implantation, the rigid inner member 101 extends longitudinally along most or all length of port 100, to function also as a rigid spine-like structure of port 100, optionally in combination with cap member 106. Inner member 101 includes a distal (front) portion 113 extending distally relative to 102 cavity, having a rounded or pointed leading edge 116 configured to facilitate or ease penetration of port 100 via the surgical opening. Port 100 may be configured such that distal portion 113 is uncovered by outer member 20 which may extend distally and transversely therefrom, although (as shown) it may be covered with a thin layer of outer member 110 such that sufficient rigid pushability is substantially uncompromised. Outer member 110 is optionally made of silicone or other flexible and elastic polymer or rubber, and is optionally extruded, casted or molded over periphery of inner member 101 or over periphery of the encapsulated core body (i.e., the structure formed by the interconnected inner member 101, septum member 104 and cap member 106), optionally within boundary of a chosen shaped mold, when forming subcutaneous port 100.

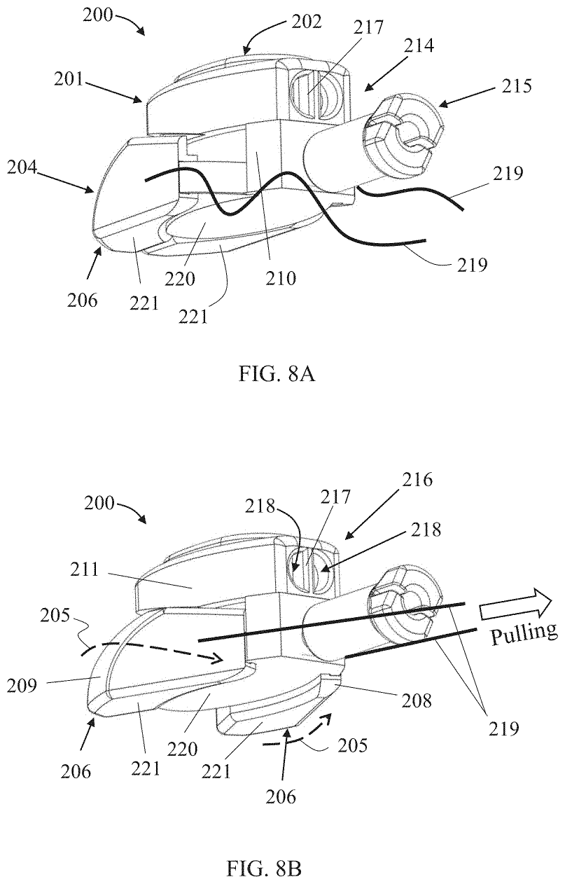

FIGS. 8A-8B illustrate axonometric views of another exemplary vascular access port 200 which includes a port body 201 and at least one port body extension 204 restrictedly movable along an at least one defined route 205 on port body 201. The at least one port body extension 204 includes a first arm 208 located right to a median plane of port body 201 and a second arm 209 located left to the median plane. Port body extensions 204, particularly first and second arms 208 and 209, are each rotatably and slidably connected to port body 201 and configured to rotate around an axis of rotation and slide on an at least one of two opposing sides of the port body 201, along routes 205, when changing from the delivery configuration to the deployed configuration. Port body 201 has an inferior portion 210 and a posterior portion 211, the posterior portion 211 is connected to a septum member 202 and the inferior portion 210 surrounds a cavity 203 that is defined by port body 201 and located below and covered by septum member 202. Inferior portion also includes a first lateral surface spanning most or all right side of inferior portion 210 and a second lateral surface spanning most or all left side of the inferior portion 210. A rear end 214 of port body 201 is coupled to a catheter connector 215 configured for connecting to a proximal end of a catheter (such as catheter 15, for example) for facilitating fluid communication between cavity 203 and a lumen of the catheter.

Vascular access port 200 is selectively changeable from a delivery configuration (as shown in FIG. 8A) to a deployed configuration (as shown in FIG. 8B) by moving first and second arms 208 and 209 along a first and a second of routs 205, respectively. When in the delivery configuration, a front portion 207 of each port body extension 204 is positioned axially distally to the port body 201. When changing to the deployed configuration, port body extensions 204 and the port body 201 are approximated along the median plane of port body 201 coincidently with laterally opposing portions 206 of port body extension 204 being parted transversely to the median plane, thereby reducing length-to-width ratio of the toggling vascular access port 200. When in the deployed configuration, the port body extensions 204 are fixedly and releasably connected to port body 201, therefore allowing selective reverting from the deployed configuration to the delivery configuration. Furthermore, rear end 214 of port body 201 is kept not covered with the port body extensions 204 also after changing to the deployed configuration, for avoiding engagement with catheter connector 215 and/or a catheter connected thereto, for example.

A port gripping portion 216 (which is optionally similar or identical in structure, function and/or dimensions to port gripping portion 21) is located on the rear end 214 of port body 201 superiorly to catheter connector 215 for allowing a user to selectively move and/or manipulate port 200 subcutaneously and in the target implantation site while avoiding engagement with catheter connector 215 and/or a catheter connected thereto, for example. A user can clamp port gripping portion 216 with medical forceps and push toggling vascular access port 200 when in the delivery configuration to the target implantation site with the medical forceps. Once in the target implantation site, port 200 can be changed to the deployed configuration by pushing port body 201 distally relative to port body extensions 204 and/or pulling port body extensions 204, such as with pulling members 219 connected to first and second arms 208 and 209 while resisting motion of port body 201 using the forceps.

Port gripping portion 216 includes a thin wall portion 217 comprising opposing lateral surfaces extending parallel to the median plane from both sides thereof, the wall portion 217 is configured for grasping and/or clamping by medical forceps including but not limited to needle holder or Kelly forceps 230, as shown in FIGS. 9A and 9B. In some embodiments, wall portion 217 is about 0.5 mm to 3 mm (optionally particularly about 1 mm to 2 mm) thick and/or about 2 mm to 5 mm (optionally particularly about 3 mm to 4 mm) wide for allowing sufficient clamping contact area and buildup of sufficient clamping, grasping or locking force from both sides of wall portion 217 using medical forceps.

Wall portion 217 can be configured as a septum dividing cavities 218 formed in rear end 214 from both sides thereof. Cavities 218 are shaped and sized to accommodate a pair of tips of the medical forceps and to allow closing motion of the pair of tips thereinside towards the wall portion and grasping of the wall portion 217 with the pair of tips from both sides thereof. FIG. 10 illustrates an alternative exemplary configuration of port gripping portion 216 having a thin wall portion 217′ similar to wall portion 217, yet not bound by cavities, rather allow greater room for forceps tips maneuverability.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. A subcutaneous port, comprising:
a port body enclosing a cavity, wherein the cavity comprises a first opening covered by a septum configured for repeated needle penetrations therethrough and a second opening configured for facilitating fluid communication between the cavity and a catheter;
wherein the port body comprises a rigid port gripping portion configured to releasably engage with a medical clamp, and wherein the port body is configured to be pushed into a subcutaneous target implantation site using the medical clamp when the medical clamp is engaged with the port gripping portion;
wherein the port gripping portion is configured to receive manual forces and/or torques from the medical clamp in at least one axis, wherein the manual forces and/or torques received at the port gripping portion are sufficient to releasably affix the medical clamp to the port gripping portion.

2. The subcutaneous port according to claim 1, wherein the port body includes a rigid port body member surrounding the cavity and/or defining the first cavity opening, the rigid port body member comprising a front portion, a rear portion, and lateral portions extending from opposing sides thereof between the front portion and the rear portion, wherein the rear portion comprises the port gripping portion.

3. The subcutaneous port according to claim 1, wherein the manual forces and/or torques received at the port gripping portion are sufficient to form or enlarge a subcutaneous void and/or passage in a body of a subject using the subcutaneous port and/or to maneuver the subcutaneous port along the subcutaneous void and/or passage, without slipping from, or releasing grip of, the port gripping portion.

4. The subcutaneous port according to claim 1, wherein the port gripping portion comprises a wall, wherein the wall comprises opposing first and second outer wall surfaces sized to accommodate clamping surfaces of the medical clamp.

5. The subcutaneous port according to claim 4, wherein the port gripping portion is configured such that the manual forces are equal to or smaller than 10 kgf and/or the manual torques are equal to or smaller than about 0.25 N*m, when the clamping surfaces of the medical clamp are oriented and spaced apart with each other to substantially match a shape and a thickness of the wall.

6. The subcutaneous port according to claim 4, wherein the port gripping portion is configured such that manually operable arms of the medical clamp are allowed to interlock when the clamping surfaces of the clamping device are oriented and spaced apart with respect to each other to match a shape and a thickness of the wall.

7. The subcutaneous port according to claim 4, wherein each of the outer wall surfaces extends vertically between lateral portions of the port body.

8. The subcutaneous port according to claim 4, wherein each of the outer wall surfaces extends horizontally between a bottom portion and a top portion of the port body.

9. The subcutaneous port according to claim 4, wherein an average or maximal thickness of the wall is between about 1 mm and about 4 mm, and/or an angle formed between the first and second outer wall surfaces is equal to or smaller than about 20°.

10. The subcutaneous port according to claim 1, wherein the second cavity opening is in juxtaposition with, and/or located inferiorly to, the port gripping portion.

11. A method comprising:
forming a surgical opening across skin layers in a subject;
creating a subcutaneous void and/or passage beneath the skin layers via the surgical opening;
clamping a port gripping portion of a subcutaneous port with a medical clamp;
pushing the subcutaneous port with the medical clamp through the surgical opening and the subcutaneous void and/or passage to a target implantation site;
releasing the medical clamp from the port gripping portion; and
removing the medical clamp from the subcutaneous void and/or passage.

12. A method according to claim 11, comprising creating or enlarging the subcutaneous void and/or passage with the medical clamp prior to the clamping.

13. A method according to claim 11, wherein the port gripping portion comprises a wall comprising opposing first and second outer wall surfaces, wherein the clamping comprises interlocking manually operable arms of the medical clamp so as to apply continuous grip against the first and second wall surfaces of the port gripping portion.

14. A method according to claim 11, comprising forming the surgical opening at an axilla of the subject.

15. A method according to claim 11, comprising inserting a first end of a catheter to vasculature of the subject via the surgical opening and coupling a second end of the catheter to the subcutaneous port to form fluid communication between a lumen of the catheter and a cavity of the subcutaneous port.

16. A method according to claim 15, wherein the first end of the catheter is inserted to the vasculature via axillary vein or jugular vein of the subject.

17. A method according to claim 15, wherein any access to the vasculature and/or across the skin layers of the subject after the forming is made directly through the surgical opening.

18. A method according to claim 11, further comprising at least one of: accessing into a vein of the subject with an access needle, inserting a wire into the vein through the access needle, removing the access needle from the vein, inserting a peel apart sheath and/or a dilator into the vein over the wire, removing the wire and/or the dilator from the vein, inserting a first end of a catheter into the vein through the peel apart sheath, and removing the peel apart sheath from the vein.

19. A method according to claim 11, wherein the vein is an axillary vein or a jugular vein.

20. A method according to claim 11, wherein the medical clamp is configured as medical forceps and/or selected from Kelly forceps, a surgical needle holder, and locking forceps.
